(19) United States
(12) Patent Application Publication
(10) Pub. No.: US 2021/0346046 A1
(43) Pub. Date: Nov. 11, 2021

(54) DISPOSABLE COVER FOR A MECHANICAL RESECTION DEVICE

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Philip CORMIER, Newburyport, MA (US); Colleen VECCHIARELLI, Londonderry, NH (US); Mathilda LUTYNSKI, Stoneham, MA (US); James YUHAN, Winchester, MA (US)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 17/246,769

(22) Filed: May 3, 2021

Related U.S. Application Data
(60) Provisional application No. 63/020,400, filed on May 5, 2020.

(51) Int. Cl. A61B 17/32

(52) U.S. Cl. CPC A61B 17/320016; A61B 2017/00991

(57) ABSTRACT

Disposable cover for a mechanical resection device. At least one example is a surgical system including: a bottom cover and a motor drive unit (MDU). The bottom cover may include: a lower trough defined by an inside surface of the bottom cover; a cover receptacle; a stationary hub rigidly coupled to the cover receptacle, the stationary hub protruding into the lower trough; and a transmission shaft defining proximal coupler, a distal coupler, and a longitudinal central axis, the transmission shaft extends through the stationary hub such that the distal coupler is in operational relationship to the cover receptacle. The MDU, disposed within the lower trough, may define a motor with a drive shaft, a handpiece receptacle on an end of the MDU, and a handpiece coupler in operational relationship to the handpiece receptacle, the handpiece receptacle telescoped over the stationary hub, and the handpiece coupler coupled to the proximal coupler.

### CROSS-REFERENCE TO RELATED APPLICATIONS

This application claims the benefit of U.S. Provisional Application Ser. No. 63/020,400 filed May 5, 2020 titled “Disposable Covering for a Wired Mechanical Resection Device,” the entire contents of which are incorporated by reference herein as if reproduced in full below.

### BACKGROUND

Arthroscopic surgical procedures may use a mechanical blade or burr to remove tissue by cutting, scraping, and/or grinding. Related-art mechanical resection devices may use suction to remove debris that is generated by the resection, and in the case of blade-type devices the suction may also pull the tissue into the cutting zone to improve resection efficiency.

In many cases, the mechanical resection devices are single use, disposable devices. However, the mechanical resection devices are operated or driven by a handpiece that is multiple-use device. Between uses the handpiece is cleaned and sterilized, such as using brushes to clean the fluid passageways within the handpiece, and subjecting the handpiece to high temperature and pressure within an autoclave. Even when proper cleaning procedures are used, there is still a chance that small pieces of tissue are not fully removed between uses. Moreover, the autoclave process is a harsh process that shortens the life of the various electromechanical components of the handpiece.

### SUMMARY

One example is a method comprising: inserting a motor drive unit (MDU) into a bottom cover, the MDU defines a handpiece receptacle on a distal end thereof and a handpiece coupler disposed within the handpiece receptacle, the bottom cover defines a stationary hub rigidly held by the bottom cover and a rotating hub configured to rotate about a longitudinal central axis, wherein the inserting the MDU telescopes the rotating hub and stationary hub into the handpiece receptacle and couples the rotating hub to the handpiece coupler; inserting a proximal end of a mechanical resection instrument into a cover receptacle defined on a distal end of the bottom cover, the cover receptacle defines a cover coupler disposed within the cover receptacle, and wherein inserting the proximal end of the mechanical resection instrument couples an inner hub of the mechanical resection instrument to the cover coupler; and resecting tissue using the mechanical resection instrument with rotational energy provided from a motor in the MDU to the inner hub of the mechanical resection instrument by way of the rotating hub of the bottom cover.

The example method may further comprise placing a top cover over at least a portion of the MDU and coupling the top cover to the bottom cover. Placing the top cover may further comprise at least partially occluding, by the top cover, both a valve handle and a tubing spigot defined by the MDU. Placing the top cover may further comprise fully occluding, by the top cover, both a valve handle and a tubing spigot defined by the MDU. Placing the top cover may further comprise leaving buttons of the MDU exposed by the top cover. Leaving the buttons of the MDU exposed may further comprise leaving the buttons exposed in a window between a distal end of the top cover and a shoulder defined between the stationary hub and an outer surface of the bottom cover.

In the example method, the MDU may include a Hall-effect sensor in operational relationship to the handpiece receptacle, and the example method may further comprise sensing a magnet field of a magnet disposed in a stationary hub of the mechanical resection instrument. Sensing the magnetic field of the magnet may further comprise sensing the magnetic field of the magnet by way of an elongate bar of ferromagnetic material, the elongate bar having a distal end disposed proximate to the cover receptacle, and a proximal end disposed on a proximal portion of the stationary hub of the bottom cover.

In the example method, the MDU may further define a fluid passageway through the MDU, the fluid passageway fluidly coupled to a spigot disposed on the proximal end and an interior volume of the handpiece receptacle, and the method may further comprise aspirating resection byproducts through a fluid passageway defined by the bottom cover and fluidly coupled to an interior volume of the cover receptacle.

Another example is a surgical system comprising: a bottom cover defining a proximal end and a distal end, the bottom cover comprising: an exterior surface; a lower trough defined by an inside surface of the bottom cover; a cover receptacle disposed at the distal end; a stationary hub rigidly coupled to the cover receptacle, the stationary hub protruding into the lower trough; a transmission shaft defining a proximal coupler on a proximal end, a distal coupler on a distal end, and a longitudinal central axis, the transmission shaft extends through the stationary hub such that the distal coupler resides in the cover receptacle, and the transmission shaft is configured to rotate about the longitudinal central axis relative to the stationary hub; and a motor drive unit (MDU) that defines a motor with a drive shaft, a handpiece receptacle on a distal end of the MDU, and handpiece coupler disposed within the handpiece receptacle and coupled to the drive shaft, the MDU disposed within the lower trough, the handpiece receptacle telescoped over the stationary hub, and the handpiece coupler of the MDU coupled to the proximal coupler of the transmission shaft.

In the example surgical system, the bottom cover may further comprise: a spigot on a proximal end of the bottom cover; a fluid passageway fluidly coupled between the spigot and an interior volume of the cover receptacle; and a valve comprising a valve member in operational relationship to the fluid passageway and a valve handle rigidly coupled to the valve member.

In the example surgical system, the bottom cover may further comprise an elongate bar of ferromagnetic material, the elongate bar defining a first end disposed on the stationary hub at a first longitudinal position relative to the longitudinal central axis, and a second end disposed on an inside surface of the cover receptacle; and the MDU may include a Hall-effect sensor disposed proximate to the handpiece receptacle at the first longitudinal position.

The example surgical system may further comprise a mechanical resection instrument. The mechanical resection instrument may comprise: an outer hub coupled to an elongate shaft, the outer hub telescoped within the cover receptacle; and a rotating hub on a proximal side of the outer hub, the rotating hub coupled the distal coupler within the cover receptacle, the rotating hub is configured to rotate relative to the outer hub about the longitudinal central axis by rotational force imparted by the distal coupler. The Hall-effect sensor may be disposed proximate to the handpiece receptacle of the MDU, the Hall-effect sensor may be disposed at a first axial position relative to the longitudinal central axis; a magnet disposed in an aperture defined in the stationary hub, the magnet disposed within the cover receptacle and at a second axial position relative to the longitudinal central axis; and an elongate bar of ferromagnetic material, the elongate bar defining a first end disposed on the stationary hub at the first axial position and a second end disposed on an inside surface of the cover receptacle at the second axial position.

The example surgical system may further comprise: a handpiece spigot on a proximal end of the MDU; a handpiece fluid passageway fluidly coupled between the handpiece spigot and an interior volume of the handpiece receptacle; a handpiece valve comprising a handpiece valve member in operational relationship to the handpiece fluid passageway and a handpiece valve handle rigidly coupled to the handpiece valve member; and a top cover coupled to the bottom cover. The top cover may comprise: an exterior surface defining a proximal end aligned with the proximal end of the bottom cover, and a distal end that resides proximally of the stationary hub; and an upper trough defined by an inside surface of the top cover, the upper trough and the lower trough define an interior volume; and wherein the top cover at least partially occludes the handpiece spigot and the handpiece valve handle. In the example surgical system further comprise: a cover spigot on a proximal end of the bottom cover; a cover fluid passageway fluidly coupled between the cover spigot and an interior volume of the cover receptacle; and a cover valve comprising a cover valve member in operational relationship to the cover fluid passageway and a cover valve handle rigidly coupled to the handpiece valve member.

The example surgical system may further comprise: a pad comprising a button, the pad and button defined by the MDU, and the pad and button proximal from the handpiece receptacle; wherein the lower trough defines a length measured parallel to the longitudinal central axis from the proximal end to a shoulder defined between the stationary hub and an outer surface of the bottom cover; and a top cover coupled to the bottom cover over the MDU, the top cover extends from the proximal end of the MDU to a proximal side of the pad and button of the MDU.

Yet another example is a device cover for a surgical device, the device cover including a bottom cover defining a proximal end and a distal end, the bottom cover may comprise: an exterior surface; a lower trough defined by an inside surface; a cover receptacle disposed at the distal end of the bottom cover; a stationary hub rigidly coupled to the cover receptacle, the stationary hub protruding into the lower trough; a transmission shaft defining proximal coupler on a proximal end, a distal coupler on a distal end, and a longitudinal central axis, the transmission shaft extends through the stationary hub such that the distal coupler resides in the cover receptacle, and the transmission shaft is configured to rotate about the longitudinal central axis and the rotation relative to the stationary hub; a spigot on a proximal end of the bottom cover; a fluid passageway fluidly coupled between the spigot and an interior volume of the cover receptacle; a valve comprising a valve member in operational relationship to the fluid passageway and a valve handle rigidly coupled to the valve member; and an elongate bar of ferromagnetic material, the elongate bar defining a first end disposed on the stationary hub, and a second end disposed on an inside surface of the cover receptacle.

In the example device may further include a top cover coupled to the bottom cover, and the top cover may comprise: an exterior surface defining a proximal end aligned with the proximal end of the bottom cover, and a distal end that resides proximally of the stationary hub; and an upper trough defined by an inside surface of the top cover, the upper trough and the lower trough define an interior volume. The lower trough may define a length measured parallel to the longitudinal central axis from the proximal end to a shoulder defined between the stationary hub and an outer surface of the bottom cover, and further including a top cover coupled to the bottom cover, and the top cover may comprise: an exterior surface defining a proximal end aligned with the proximal end of the bottom cover, and a distal end, and a length, and wherein the length of the top cover is less than the length of the lower trough; and an upper trough defined by an inside surface of the top cover, the upper trough and the lower trough define an interior volume. The valve member and valve handle together may circumscribe the longitudinal central axis.

### BRIEF DESCRIPTION OF THE DRAWINGS

For a detailed description of example embodiments, reference will now be made to the accompanying drawings in which:

FIG. 1 shows a resection system in accordance with at least some embodiments;

FIG. 2 shows a perspective view of a wand in accordance with at least some embodiments;

FIG. 3 shows a cross-sectional view of a motor drive unit (MDU) in accordance with at least some embodiments;

FIG. 4 shows a simplified cross-sectional side-elevation view of a MDU and attached mechanical resection instrument, in accordance with at least some embodiments;

FIG. 5 shows an exploded perspective view of resection system in accordance with at least some embodiments;

FIG. 6 shows an exploded perspective view of the cover system in accordance with at least some embodiments;

FIG. 7 shows a cross-sectional view of the cover system with a MDU disposed therein, and in accordance with at least some embodiments;

FIG. 8 shows a cross-sectional view of a portion of the cover system in greater detail, and in accordance with at least some embodiments;

FIG. 9 shows an exploded perspective view of additional components of the cover system, and in accordance with at least some embodiments;

FIG. 10 shows a partial perspective view of the cover system showing placement of the elongate bar in accordance with at least some embodiments;

FIG. 11 shows a partial perspective view of a portion of the cover system showing placement of the elongate bars in accordance with at least some embodiments; and

FIG. 12 shows a method in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

“Mechanical resection instrument” shall mean an instrument that removes tissue (including bone) by way of cutting, scraping, and/or grinding. Instruments that remove tissue electrically (i.e., by flowing electrical current through the tissue to desiccate the tissue, or by exposing the tissue to plasma) shall not be considered to be mechanical resection instruments.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to a disposable cover for a surgical device, and methods of use. More particularly, various examples are directed to a disposable cover for a motor drive unit (MDU) to which a mechanical resection instrument is connected, and from which the mechanical resection instrument is provided rotational energy used for resecting tissue. Example disposable covers include a bottom cover that has a reproduction of an outer hub and an inner hub of a mechanical resection instrument, and further has a reproduction of a receptacle of the MDU. The MDU couples to the reproduction of the outer hub and the inner hub, and the mechanical resection instrument couples to the reproduction of the receptacle. Thus, the MDU provides rotational energy to the mechanical resection instrument through the reproduced or duplicated elements. In still further example cases, the disposable covers include a fluid passageway that extends between a spigot on a proximal end of the disposable cover and the reproduction of the receptacle. Aspiration of resection byproducts may thus take place through the fluid passageway of the disposable cover to the exclusion of a fluid passageway defined by the MDU. Thus, the disposable cover shields the MDU from exposure to fluids and tissue which obviates the need to perform extensive cleaning and autoclave process for the MDU. The specification first turns to an example system, without a disposable cover, to orient the reader.

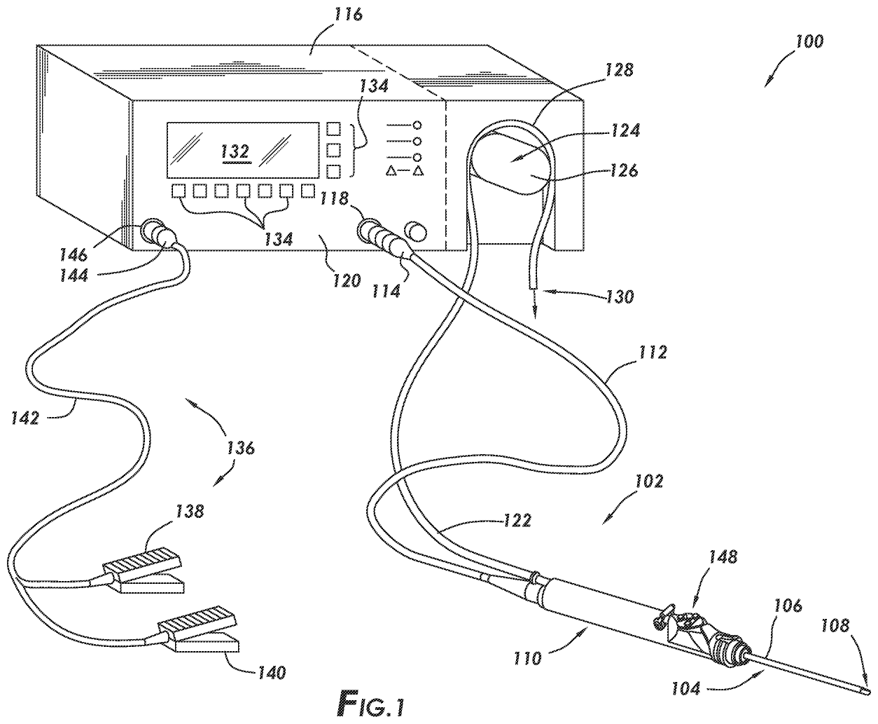

FIG. 1 shows a resection system in accordance with at least some embodiments. In particular, the resection system 100 comprises a wand 102. The wand 102 includes a mechanical resection instrument 104 that comprises an elongate shaft 106 that defines distal end 108. Further, the wand 102 comprises a handpiece or MDU 110. The wand 102 further comprises a flexible multi-conductor cable 112 housing one or more electrical leads (not specifically shown in FIG. 1), and the flexible multi-conductor cable 112 terminates in a wand connector 114. As shown in FIG. 1, the wand 102 couples to a resection controller 116, such as by a controller connector 118 on an outer surface of the enclosure 120 (in the illustrative case of FIG. 1, the front surface of the enclosure 120).

Though not visible in the view of FIG. 1, in some embodiments the wand 102 has one or more internal aspiration channels or fluid passageways. The fluid passageway of the wand 102 couples to hose or flexible tubular member 122 used to provide suction or aspiration at the distal end 108 of the wand 102. In accordance with example embodiments, the flexible tubular member 122 couples to a peristaltic pump 124, which peristaltic pump 124 is illustratively shown as an integral component with the resection controller 116 (i.e., residing at least partially within the enclosure 120 of the resection controller 116). In other embodiments, an enclosure for the peristaltic pump 124 may be separate from the enclosure 120 for the resection controller 116 (as shown by dashed lines in the figure), but in any event the peristaltic pump 124 is operatively coupled to the resection controller 116.

The peristaltic pump 124 comprises a rotor portion 126 (hereafter just “rotor 126”) as well as a stator portion 128 (hereafter just “stator 128”). The flexible tubular member 122 couples within the peristaltic pump 124 between the rotor 126 and the stator 128, and movement of the rotor 126 against the flexible tubular member 122 causes fluid movement toward the discharge 130. While the illustrative peristaltic pump 124 is shown with a two-head rotor 126, other types of peristaltic pumps 124 may be used (e.g., a five-head peristaltic pump, linear peristaltic pump). In the context of the various embodiments, the peristaltic pump 124 creates a volume-controlled aspiration from a cavity or surgical field at the distal end 108 of the wand 102 (the surgical field not specifically shown), with the outflow rate based on a speed of the rotor 126, as commanded by the resection controller 116. In yet still other cases, the suction or vacuum to enable the aspiration may be provided from any suitable source, such as a vacuum connection available in most surgical rooms.

Still referring to FIG. 1, a display device or interface device 132 is visible through the enclosure 120 of the resection controller 116, and in some embodiments a user may select operational modes of the resection controller 116 by way of the interface device 132 and related buttons 134. For example, using one or more of the buttons 134 the surgeon may select a rotational mode of the rotating portion of the mechanical resection instrument 104 (the rotating portion discussed more below). As another example, using one or more of the buttons 134 the surgeon may select an aggressiveness of the outflow control through the wand 102.

In some embodiments the resection system 100 also comprises a foot pedal assembly 136. The foot pedal assembly 136 may comprise one or more foot pedal devices 138 and 140, a flexible multi-conductor cable 142, and a pedal connector 144. While only two foot pedal devices 138 and 140 are shown, one or more pedal devices may be implemented. In other cases, a single foot pedal assembly may be used, where the foot pedal assembly includes two or more foot pedals. The enclosure 120 of the resection controller 116 may comprise a corresponding connector 146 that couples to the pedal connector 144. A clinician may use the foot pedal assembly 136 to control various aspects of the resection controller 116. For example, foot pedal device 138 may be used for on-off control of the motor within the wand 102. Further, foot pedal device 140 may be used to control and/or set rotational mode of the rotating portion of the mechanical resection instrument 104. Alternatively, control of the various operational or performance aspects of the resection controller 116 may be activated by selectively depressing buttons 148 located on the MDU 110 of the wand 102.

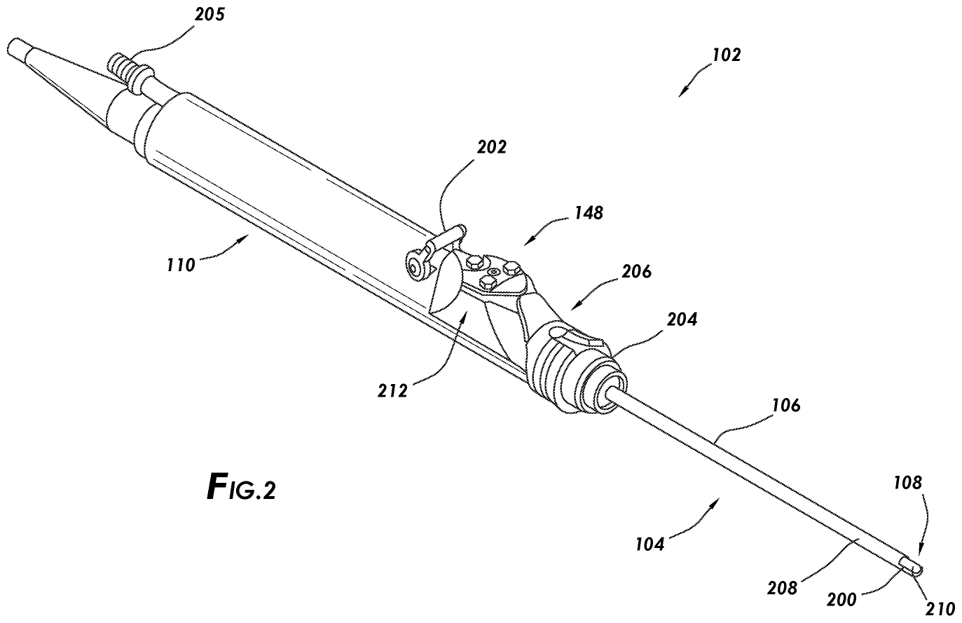

FIG. 2 shows a perspective view of a wand 102 in accordance with at least some embodiments. In particular, visible in FIG. 2 is the example MDU 110 as well as the example mechanical resection instrument 104. In the example wand 102 of FIG. 2, the mechanical resection instrument 104 is a blade-type device where the elongate shaft 106 is defined by an outer tube, and telescoped within the outer tube is an inner tube 210. At the distal end 108 a cutting window 200 is provided through the outer tube 208, and the example inner tube 210 is visible through the cutting window. The inner tube 210 is telescoped within and concentrically disposed within the outer tube 208, and the inner tube has a corresponding cutting window (not visible in FIG. 2). When the cutting windows of the outer tube 208 and the inner tube 210 align, tissue is drawn into the cutting window 200, and the interaction of cutting surfaces of the outer tube 208 and inner tube 210 cuts the tissue. In the case of the mechanical resection instrument being a blade-type device, suction provided by the peristaltic pump 124 or other vacuum source draws fluid and resection byproducts through the outer tube 208; and more particularly, the peristaltic pump 124 or other vacuum source draws fluid and resection byproducts through the inside diameter of the inner tube 210.

FIG. 2 further shows that the MDU 110 may implement the valve handle 202. The valve handle 202 is rotationally coupled to an outside surface of the MDU 110. The valve handle 202 is attached to a valve member (not visible in FIG. 2) within the MDU 110. More specifically, the valve member is disposed within the fluid passageway defined within the MDU 110, and the fluid passageway is fluidly coupled to the hose connector or spigot 205. When the wand 102 is used in the configuration shown in FIG. 2, the clinician actuates the valve handle to modulate the aspiration through the wand 102.

Also visible in FIG. 2 are the buttons 148 that the clinician may use to control various aspects of the operation of the wand 102 (e.g., speed of rotation of the inner tube, direction of rotation, rotational mode, aggressiveness of aspiration through the wand 102). In particular, the example MDU 110 defines a plateau or pad 212 having an upper surface. The example buttons 148 may protrude upwardly from the pad 212. Considered along the longitudinal central axis of the MDU 110, the pad 212 and buttons 148 reside between the valve handle 202 and the receptacle defined by the distal end 206 of the MDU.

In many cases, the mechanical resection instrument 104 is a single-use item that is used for a particular surgical procedure, and then discarded. By contrast, the MDU 110 may be cleaned, sterilized, and reused for multiple surgical procedures. Many different mechanical resection instruments may be coupled to the MDU 110 (and thus coupled to the resection controller 116 (FIG. 1)). The different mechanical resection instruments include not only the categories of blade-type and burr-type devices, but also variations within each category. For example, there may be many blade-type devices from which a surgeon may choose, such as blade-type devices having varying outside diameters of the elongate shaft 106, varying axial lengths of the elongate shaft 106 (i.e., lengths measured parallel to a longitudinal axis through the inside diameter of the outer tube that defines the elongate shaft 106), and varying aggressiveness of cutting surfaces (e.g., smooth, toothed). Regardless of the mechanical resection instrument selected (and, in fact, multiple instruments can be used in any one surgical procedure), each mechanical resection instrument 104 includes a stationary hub or outer hub 204 designed and constructed to couple to the receptacle defined and disposed on a distal end 206 of the MDU 110. When the outer hub 204 is mechanically coupled within the receptacle of the MDU 110 as shown in FIG. 2, the flow pathway within the mechanical resection instrument 104 is fluidly coupled to the fluid passageway through the MDU 110, and the rotating portion (e.g., a rotating hub or inner hub coupled to the inner tube 210) of the mechanical resection instrument 104 is mechanically coupled to the motor within the MDU 110.

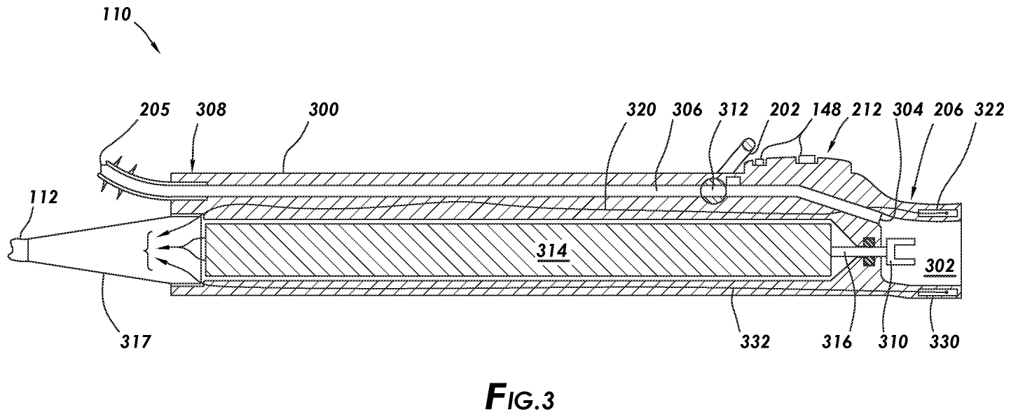

FIG. 3 shows a cross-sectional view of the MDU 110 in accordance with at least some embodiments. In particular, the example MDU 110 comprises an outer casing 300 that defines an outside surface. Exposed on the outside surface of the outer casing 300, and in particular on the pad 212, are the buttons 148 discussed above. The distal end 206 of the MDU 110 includes a receptacle 302 into which the outer hub 204 (FIG. 2) is inserted when the mechanical resection instrument 104 (FIGS. 1 and 2) is mated with the MDU 110. In the example system, the receptacle 302 and outer hub 204 are constructed such that the outer hub 204 telescopes into the inside diameter of the receptacle 302. Within the receptacle 302 is defined a hole or aperture 304 that leads to the fluid passageway 306 through the MDU 110. At the proximal end 308 of the MDU 110 resides the spigot 205 that is fluidly coupled to the fluid passageway 306. The spigot 205 enables connection to the flexible tubular member 122 (FIG. 1).

FIG. 3 also shows the valve handle 202 rotationally coupled to the outside surface of the outer casing 300 of the MDU 110. The valve handle 202 is rigidly coupled to an example internal valve member 312 in operational relationship with the fluid passageway 306. In one rotational orientation of the valve handle 202 and thus valve member 312, and as shown in FIG. 3, the valve member 312 presents no impediment to the flow of the fluids and/or resection byproducts through the fluid passageway 306. In a second rotational orientation of the valve handle 202 and thus valve member 312 (the second rotational orientation not specifically shown), the valve member 312 may be oriented such that some impediment to the flow of the fluids and/or resection byproducts through the fluid passageway 306 is present.

The example MDU 110 further comprises a motor 314 that defines a drive shaft 316. The motor 314 is disposed within the outer casing 300 of the MDU 110, and the drive shaft 316 is in operational relationship to the receptacle 302. A handpiece coupler 310 is coupled to the drive shaft 316, and the handpiece coupler 310 is disposed within the receptacle 302. The example handpiece coupler 310 is shown as a drive fork designed and constructed to mate with a drive tang defined by the inner hub of a mechanical resection instruction; however, any suitable handpiece coupler 310 may be used. When the outer hub 204 (not shown in FIG. 3) of a mechanical resection instrument 104 is coupled to the MDU 110, the rotating portion of the mechanical resection instrument (e.g., the inner hub) is mechanically coupled to the drive shaft 316 by way of the handpiece coupler 310 such that the motor 314 can control rotation of the rotating portion. While FIG. 3 shows the drive shaft 316 extending into the receptacle 302, in other cases various gears may be disposed between the drive shaft 316 and the rotating portion of the mechanical resection instrument to set the relationship between drive shaft 316 rotational speed and rotational speed of the rotating portion of the mechanical resection instrument 104. Assuming the motor 314 is an electrical motor for purposes of the further disclosure, electrical leads that couple to windings of the motor 314 feed through the connector 317 and into the flexible multi-conductor cable 112.

The example MDU 110 further comprises one or more magnetic field sensors in operational relationship to the receptacle 302. In the example MDU 110 of FIG. 3, two magnetic field sensors in the example form of Hall-effect sensors are used, one Hall-effect sensor 322 on an “upper” side of the MDU 110 (e.g., on the same side as the buttons 148), and one Hall-effect sensor 330 on the “bottom” side, opposite the buttons 148. The example Hall-effect sensor 322 is coupled to the resection controller 116 by way of the leads 320. While the example Hall-effect sensor 322 is shown disposed in a side wall of the receptacle 302, the Hall-effect sensor 322 may be placed at any suitable location for reading magnetic field strength associated with magnets of the mechanical resection instrument, as discussed more below. Similarly, the Hall-effect sensor 330 is coupled to the resection controller 116 by way of the leads 332. While the example Hall-effect sensor 330 is shown disposed in a side wall of the receptacle 302, the Hall-effect sensor 330 may be placed at any suitable location for reading magnetic field strength associated with the magnets of the mechanical resection instrument. In the example case of MDU 110 of FIG. 3, the Hall-effect sensors 322 and 330 are placed on opposite sides of the receptacle 302 (e.g., 180 rotational degrees apart relative to the long central axis of the MDU 110), but such placement is not strictly required.

Each Hall-effect sensor 322 and 330 is a magnetic field sensor that produces a voltage output proportional to the magnetic field strength in proximity of the sensor. In some cases, the Hall-effect sensors are used in Boolean sense—determining the presence or absence of a magnet in proximity to the Hall-effect sensor. In other cases, the voltage output of each Hall-effect sensor is used in an analog sense. That is, a voltage output from one or both Hall-effect sensors may not only indicate the presence of a mechanical resection instrument, but the magnitude of the voltage output may also convey certain information.

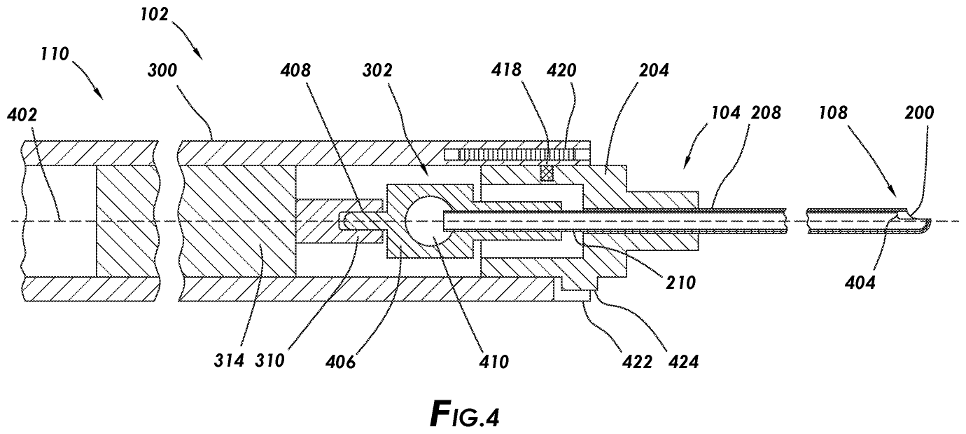

FIG. 4 shows a simplified cross-sectional side-elevation view of the MDU 110 and attached mechanical resection instrument 104, in accordance with at least some embodiments. In particular, FIG. 4 shows a portion of the outer casing 300 that defines the receptacle 302. The receptacle 302 defines an inside diameter into which the example outer hub 204 is telescoped. The outer hub 204 is rigidly coupled to the outer tube 208, and the outer tube 208 includes the cutting window 200 on the distal end 108. The example mechanical resection instrument 104 further includes the inner tube 210 concentrically disposed within the outer tube 208. The inner tube 210 and outer tube 208 share a longitudinal central axis 402, and in the example cases the longitudinal central axis 402 is also coaxial with the longitudinal central axis of the drive shaft 316 of the MDU 110. The inner tube 210 likewise defines a cutting window 404, and in the view of FIG. 4 the cutting window 404 of the inner tube 400 is aligned with the cutting window 200 of the outer tube 208. In particular, in the view of FIG. 4 the cutting window 404 of the inner tube 210 is fully aligned with the cutting window 200 of the outer tube 208 such that the effective size of the aperture into the inside diameter of the inner tube 210 is at its peak.

On the proximal end of the mechanical resection instrument 104 resides a rotating hub or inner hub 406 disposed at least partially within an inside diameter of the outer hub 204. The inner hub 406 is rigidly coupled to the inner tube 210 such that rotation of the inner hub 406 about the longitudinal central axis 402 likewise rotates the inner tube 210 about the longitudinal central axis 402. The example inner hub 406 defines an instrument coupler 408 that mates with the handpiece coupler 310. The instrument coupler 408 is shown in the example form of a tab or tang that fits within the handpiece coupler 310 in the example form of a drive fork. The inner hub 406 further defines a rotating slough chamber 410 fluidly coupled to the inner tube 210. Fluid and resection byproducts may thus be drawn through the cutting window 404, through the inside diameter of the inner tube 400, through the rotating slough chamber 410, and then through the fluid passageway 306 (not shown in FIG. 4) of the MDU 110.

FIG. 4 further shows the outer hub 204 defines a bore (e.g., blind bore, through bore) into which a magnet 418 is placed. The magnet 418 is held in its bore in any suitable way. For example, the magnet 418 may be press-fit within the inside diameter of the bore. In other cases, the magnet 418 may be held in by use of an adhesive. In the case of the magnet 418, if there is sufficient space within the outer hub 204, the magnet 418 may be adhered to the inside diameter of the outer hub 204 and thus not reside within a bore.

Still referring to FIG. 4, the sidewall that defines the receptacle 302 hosts the magnetic field sensor 420 that is representative of either Hall-effect sensor 322 or Hall-effect sensor 330 of FIG. 3. On the opposite side of the MDU 110 from the magnetic field sensor 420 is a slot 422. The example outer hub 204 defines a protrusion or tab 424 that extends outward from an outside surface of the outer hub 204. When the outer hub 204 is telescoped within the receptacle 302, the tab 424 telescopes within the slot 422. For an example MDU 110 that has two slots, it follows that the outer hub 204 may telescope into the receptacle 302 in one of two distinct rotational orientations about the longitudinal central axis 402. In example embodiments, the radial location of the tab 424 relative to the longitudinal central axis 402 of the mechanical resection instrument 104 is designed and constructed to place the magnet 418 associated with the outer hub 204 proximate to the location of the associated Hall-effect sensor. In the example of FIG. 4, the slot 422 places the magnet 418 proximate to the representative magnetic field sensor 420.

In accordance with example embodiments the resection controller 116 is designed and constructed to sense, by way of the representative magnetic field sensor 420, the magnet 418 coupled to the outer hub 204, and determine a location of the cutting window 200 of the outer tube 208 based on the magnetic field strength. In some cases, the resection controller 116 may determine an additional parameter from the magnitude of the magnetic field strength, such as identity of the mechanical resection instrument 104, rotational mode of the mechanical resection instrument 104 (e.g., forward rotation mode, reverse rotational mode oscillation mode), and designed speed of the rotation, to name a few.

Returning to FIG. 2. The discussion to this point has assumed that the mechanical resection instrument 104 is directly coupled to the MDU 110 for use during a surgical procedure. When the mechanical resection instrument 104 is directly coupled to the MDU 110 for the surgical procedure, the clinician may directly touch and handle the MDU 110, and the resection byproducts may be aspirated through the fluid passageway 306 (FIG. 3) of the MDU 110. After the procedure, the mechanical resection instrument 104 is discarded and the MDU 110 is subject to cleaning and sterilization process. For example, a technician may use a brush to clean the fluid passageway 306 and the interior surfaces of the receptacle 302 (not necessarily the same brush). Moreover, the external surfaces of the MDU may be physically cleaned with a cloth or brush soaked in a cleaning solution. Thereafter, the MDU 110 may be placed in an autoclave and subjected to high temperature (e.g., at least 121° C.) and pressure (e.g., at least 15 PSIG) for extended period of time (e.g., 15 to 30 minutes, or more). Moreover, the MDU 110 may need to be allowed to cool for an extended period of time before use to allow the internal components (e.g., motor 314 (FIG. 3)) to return to a temperature below operating temperature. The process is thus labor intensive and takes an extended period of time. The particular MDU 110 may not be available for use in another surgical procedures for several hours. Even when proper cleaning procedures are used, there is still a chance that small pieces of tissue are not fully removed between uses, thus increasing the possibility of cross-contamination between patients. Moreover, the autoclave process is harsh process that shortens the life of the various electromechanical components of the MDU 110.

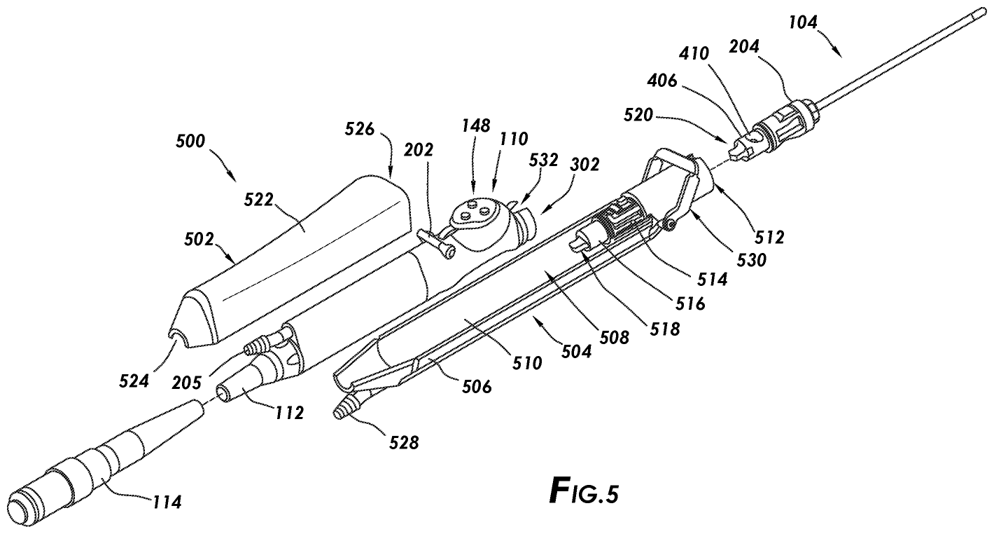

FIG. 5 shows an exploded perspective view of a surgical system or resection system in accordance with at least some embodiments. In particular, visible in FIG. 5 is the MDU 110 and a mechanical resection instrument 104. In order to shield the MDU 110 from fluids, blood, and resection byproducts, and thus obviate the need for extensive cleaning between uses, the example resection system further includes a cover system 500. The example cover system 500 comprises a top cover 502 and a bottom cover 504. Each will be addressed in turn, starting with the bottom cover 504.

The example bottom cover 504 defines an exterior surface 506. In example cases, portions of the exterior surface 506 at least partially replicate the shape the bottom surface of the MDU 110, though the overall dimensions are slightly larger to accommodate the MDU 110. The bottom cover 504 further includes a channel or lower trough 508 defined by an inside surface 510 of the bottom cover 504. The distal end of the bottom cover 504 defines a cover receptacle 512. The cover receptacle 512 is designed and constructed such that the proximal end of the mechanical resection instrument 104 (e.g., the outer hub 204 and inner hub 406) telescope within the cover receptacle 512 and hold the mechanical resection instrument 104 in place during resection procedures. Thus, the cover receptacle 512 is a substantial duplicate of the receptacle 302 of the MDU 110, with exceptions discussed more below.

In assembling the resection system for use, the proximal end of a mechanical resection instrument 104 is inserted into the receptacle 512 defined on a distal end of the bottom cover 504. The receptacle 512 defines a distal coupler (discussed more below) disposed within and/or in operational relationship to the receptacle 512, and wherein inserting the proximal end of the mechanical resection instrument 104 couples an instrument coupler 520, defined by the inner hub 406, to the distal coupler within the receptacle 512. The example instrument coupler 520 is shown as a tab or tang, and it follows the distal coupler will have a complementary structure, such as a channel or fork.

Still referring to FIG. 5, the example bottom cover 504 further defines a stationary hub 514 rigidly coupled to the cover receptacle 512, and with the stationary hub 514 protruding into the lower trough 508. The example bottom cover 504 further includes a rotating hub 516 in operational relationship to the stationary hub 514, and also protruding into the lower trough 508. In particular, the rotating hub 516 is associated with a drive shaft (discussed more below) that transfers rotational energy from the MDU 110 to the distal coupler (discussed more below) within the receptacle 512. The distal coupler, in turn, transfers the rotational energy to the inner hub 406 of the mechanical resection instrument 104. Thus, the rotating hub 516 rotates about a longitudinal central axis, and the rotation is relative to the stationary hub 514 and the balance of the bottom cover 504. The stationary hub 514 and rotating hub 516 are designed and constructed such that the receptacle 302 of the MDU 110 telescopes over the stationary hub 514 and the rotating hub 516, rather than the receptacle 302 of the MDU 110 coupling directly to the mechanical resection instrument 104. Thus, the stationary hub 514 and rotating hub 516 are substantial duplicates of the outer hub 204 and inner hub 406, respectively. For reasons that will become clearer based on the discussion below, the rotating hub 516 does not have and thus does not duplicate the rotating slough chamber 410 of the inner hub 406

In assembling the resection system for use, the MDU 110 is inserted into the bottom cover 504. More particularly, the receptacle 302 of MDU 110 is telescoped over the rotating hub 516 and stationary hub 514 as the MDU 110 is placed in the lower trough 508. For future discussion, notice the notch 532 defined on the upper surface of the receptacle 302. Inserting the MDU 110 into the bottom cover 504 couples the handpiece coupler 310 (FIG. 3) to a proximal coupler 518 defined on the proximal end of the rotating hub 516. In the example system of FIG. 5, the proximal coupler 518 is a duplicate structure to the instrument coupler 520, and thus in the example shown the proximal coupler 518 is shown as a tab or tang.

Still referring to FIG. 5, the example resection system further comprises the top cover 502. The example top cover 502 includes an exterior surface 522 defining a proximal end 524 and a distal end 526. When the top cover 502 is coupled to the bottom cover 504, the proximal end 524 is aligned with a proximal end of the bottom cover 504, and the distal end 526 resides proximally of the stationary hub 514. Though discussed in greater detail below, the top cover 502 is designed and constructed to fully or partially cover or occlude the spigot 205 of the MDU 110, and to fully or partially cover or occlude the valve handle 202, while leaving the buttons 148 exposed for use by the clinician. To that end, the example top cover 502 defines an upper trough (not visible in FIG. 5), where the upper trough is defined by an inside surface of the top cover 502. When the top cover 502 and bottom cover 504 are coupled together, the upper trough and the lower trough 508 define an interior volume in which resides a majority of the MDU 110. That is, in example systems only the buttons 148 on the pad 212 (FIG. 2) and the upper and outside portions of the receptacle 302 are exposed.

Still referring to FIG. 5, the example bottom cover 504 further defines a tube connection or spigot 528 on a proximal end of the bottom cover 504. Though not visible in the view of FIG. 5, the bottom cover 504 further defines a fluid passageway fluidly coupled between the spigot 528 and an interior volume of the receptacle 512 of the bottom cover 504. The example bottom cover 504 further defines a valve comprising a valve member (not visible) in operational relationship to the fluid passageway, and a valve handle 530 rigidly coupled to the valve member.

In assembling the resection system for use, the spigot 205 and valve handle 202 of the MDU 110 are occluded by the top cover 502, and thus not used during the resection procedure. Rather, the peristaltic pump 124 or other vacuum source is coupled to the spigot 528 and aspiration is provided along the fluid passageway of the bottom cover 504. Moreover, during resection the aspiration may be modulated by the clinician interacting with the valve handle 530 of the bottom cover 504 rather than the valve handle 202 of the MDU 110. Stated otherwise, the spigot 205, fluid passageway through the MDU 110, and receptacle 302 of the MDU 110 do not come in contact with the fluid and resection byproducts, which obviates the need for the enhanced cleaning of the MDU 110. Rather, the fluid and resection byproducts only contact the spigot 528, fluid passageway through the bottom cover 504, and the receptacle 512 of the bottom cover 504, which need not be cleaned as such is a disposable product.

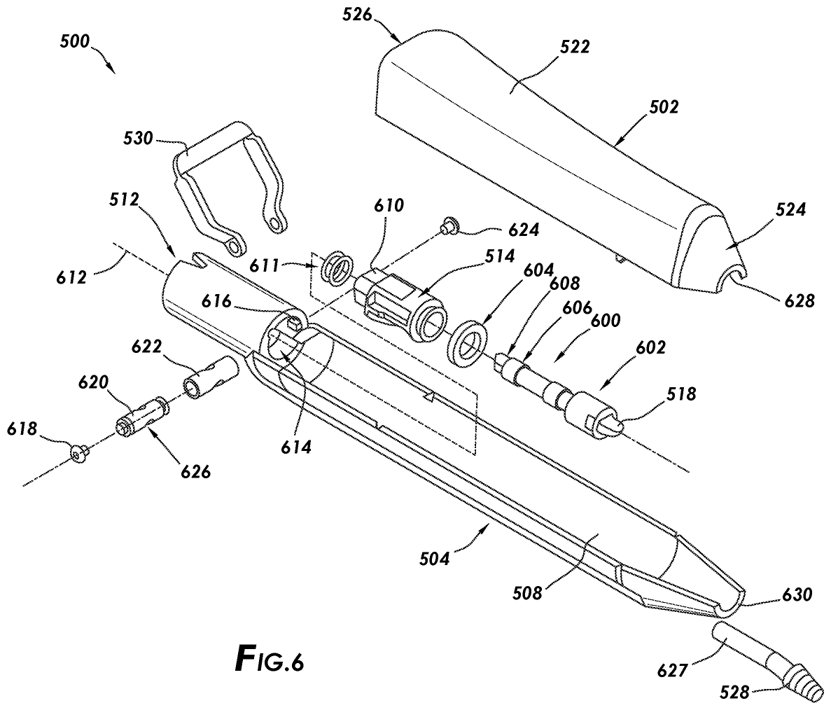

FIG. 6 shows an exploded perspective view of the cover system 500 in accordance with at least some embodiments. In particular, FIG. 6 shows the top cover 502 and bottom cover 504. FIG. 6 further shows in greater detail the various components that couple to the MDU 110 (FIG. 1), couple to the mechanical resection instrument 104 (FIG. 1), and transfer rotational energy from the MDU 110 to the mechanical resection instrument 104. More particularly still, FIG. 6 shows a transmission shaft 600 that defines proximal coupler 518 on a proximal end. In the example case of FIG. 6, the proximal coupler is shown as a tab or tang. Moving distally, the example transmission shaft defines a proximal seal area 602. The example proximal seal area 602 defines a cylindrical exterior surface designed and constructed to interact with and seal against the seal 604; however, the proximal seal area may take any suitable form depending on the nature of the proximal seals. Still moving distally, the example transmission shaft 600 further defines a distal seal area 606. The example distal seal area 606 defines a cylindrical exterior surface designed and constructed to interact with and seal against seals in the example form of O-rings 611; however, the distal seal area 606 may take any suitable form. Moving distally again, the example transmission shaft 600 further defines a distal coupler 608 on a distal end. In the example case of FIG. 6, the distal coupler 608 is shown as a channel or fork configured to interact with the tab or tang defined by a mechanical resection instrument 104.

The example transmission shaft 600 defines a longitudinal central axis 612, and the transmission shaft 600 is telescoped along the longitudinal central axis 612 through the seal 604 and the stationary hub 514. That is, the example stationary hub 514 defines a through bore through which the transmission shaft 600 telescopes. The example stationary hub 514 defines a proximal shoulder area on the proximal end and to which the example seal 604 couples, and again the seal 604 seals against the proximal seal area 602 of the transmission shaft 600. On the opposite end, the example stationary hub 514 defines a distal shoulder area 610 having one or more features designed to hold the stationary hub 514 against rotation relative to the balance of the bottom cover 504. In the example shown, the distal shoulder area 610 defines a polygonal structure in which at least one outside dimension (measured perpendicularly to the longitudinal central axis 612 of the transmission shaft 600) is smaller than abutting medial portion of the stationary hub 514, thus forming the shoulder that limits distal movement of the stationary hub 514 during assembly. Moreover, in example systems the outside dimensions of the distal shoulder area 610 are designed and constructed to press fit within a complementary area on the bottom cover (the complementary area discussed more below) and form a fluid-tight seal.

The example O-rings 611 are placed within respective annular grooves defined on an inside diameter of the through bore of the stationary hub 514. As noted above, when assembled the O-rings 611 seal against the distal seal area 606 of the transmission shaft 600. While O-rings 611 are shown, any suitable seal may be used for the distal seal. The combination of the distal seal (e.g., O-rings 611) and the proximal seal (e.g., seal 604) together form a fluid-tight barrier such that the MDU is not exposed to fluids or resection byproducts. When assembled, the distal coupler 608 may disposed distally of the distal end of the stationary hub 514, and thus the distal coupler 608 may reside within the receptacle 512 defined by the bottom cover 504 and/or may reside in operational relationship to the receptacle 512. The transmission shaft 600 may thus rotate about the longitudinal central axis 612 relative to the stationary hub 514.

Still referring to FIG. 6, the example bottom cover 504 again defines the lower trough 508 in operational relationship to the receptacle 512. In particular, the bottom cover 504 defines a through bore 614. The through bore 614 has a proximal portion that the defines an inside shape complementary to that of the distal shoulder area 610 such that the distal shoulder area 610 telescopes into the through bore 614 along the longitudinal central axis 612. In operational relationship to the through bore 614, and the stationary hub 514 when assembled, is a ridge or tab 616 that protrudes upward away from the longitudinal central axis 612. The tab 616 is designed and constructed to fit within the notch 532 (FIG. 5) defined by the MDU 110 to help align the MDU 110 within the bottom cover 504 and to reduce or prevent rotation of the MDU 110 relative to the bottom cover 504 about the longitudinal central axis 612.

The example resection system of FIG. 6 further includes an exploded view of the various components that make up the valve associated with the bottom cover 504. In particular, the example valve comprises a fastener 618, a valve member 620, a valve sleeve 622, a fastener 624, and the valve handle 530. The bottom cover 504 defines a through bore (not visible in FIG. 6) that extends across the bottom cover 504 in a direction perpendicular to a longitudinal central axis of the fluid passageway (also not visible in FIG. 6). The valve sleeve telescopes through the through bore and acts as a bearing surface and seal for the valve. The valve member 620 telescopes through the valve sleeve 622, and the valve member 620 has a flow bore 626. The orientation of the flow bore 626 controls the flow of aspirated fluid and resection byproducts through the valve and thus the fluid passageway. The valve handle 530 defines an inverted “U”-shaped structure, and the distal end of the legs of the valve handle 530 are each respectively coupled to opposite sides of the valve member 620 by way of respective fasteners 618 and 624. Thus, the valve handle 530 is rigidly coupled to the valve member 620. When assembled, the example valve handle 530 and valve member 620 considered together circumscribe the longitudinal central axis 612. FIG. 6 also shows that in example systems the spigot 528 may fluidly couple to the fluid passageway through the bottom cover 504 by way of a tubing stub or nipple 627.

FIG. 6 further shows the top cover 502 in perspective view. Visible in FIG. 6 are the proximal end 524, the distal end 526, and the exterior surface 522. The example proximal end 524 defines a tapered section terminated proximally in a cutout or notch 628. Similarly, the proximal end of the bottom cover 504 defines a tapered section with a cutout or notch 630. When the top cover 502 is coupled to the bottom cover 504, the notch 628 and notch 630 enable the flexible multi-conductor cable 112 (FIG. 1) to protrude from the cover system.

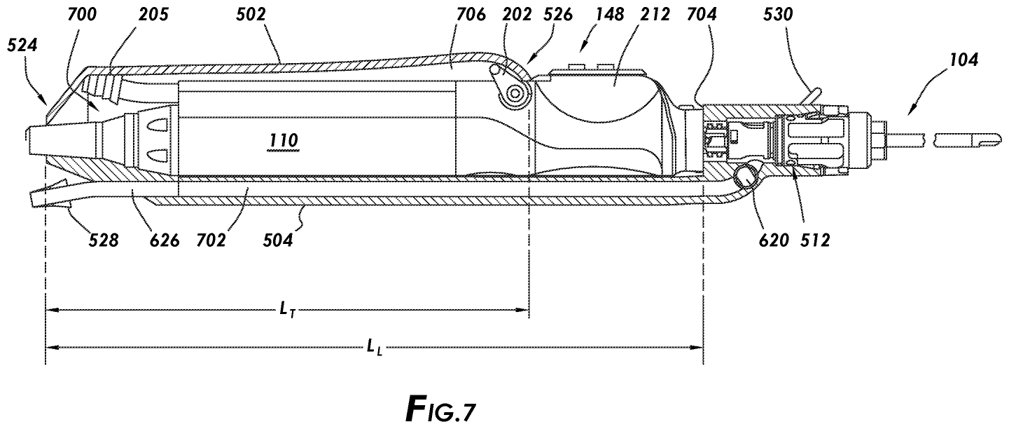

FIG. 7 shows a cross-sectional view of the cover system in accordance with at least some embodiments. In particular, FIG. 7 shows the top cover 502 coupled to the bottom cover 504, and with the MDU 110 (not shown in cross-section) disposed within the internal volume 700 defined by the top cover 502 and bottom cover 504. Moreover, FIG. 7 shows the mechanical resection instrument 104 (not shown in cross-section) coupled to the receptacle 512 defined by the bottom cover 504. Further visible in FIG. 7 is the fluid passageway 702 defined by the bottom cover 504. In particular, the spigot 528 resides at the proximal end of the bottom cover 504. The fluid passageway 702 is fluidly coupled between the spigot 528 and an interior volume of the cover receptacle 512. The valve comprising the valve member 620 is disposed in operational relationship to the fluid passageway 702, and the valve handle 530 is rigidly coupled to the valve member 620. In operation, fluid and resection byproducts are aspirated from within the interior volume of the cover receptacle 512, through the valve member 620, and then through the fluid passageway 702 defined by the bottom cover 504. Moreover, the clinician may modulate the aspiration by interacting with the valve handle 530, which interaction changes the rotational orientation of the valve member 620.

The lower trough of the bottom cover 504 defines a length L_{L} (measured parallel to the longitudinal central axis of the system) from the proximal end to a shoulder 704 defined between the stationary hub (not visible) and an outer surface of the bottom cover 504. The top cover 502 is coupled to the bottom cover 504. In the example system the proximal end 524 of the top cover 502 is aligned, along the longitudinal central axis of the system, with the proximal end of the bottom cover 504. Moreover, the distal end 526 of the top cover 502 resides on the proximal side of the pad 212 and buttons 148 of the MDU 110. That is, the top cover 502 defines a length L_{T} measured parallel to the longitudinal central axis from the proximal end 524 to the distal end 526, and wherein the length L_{T} is shorter than the length L_{L} such that the pad 212 and buttons 148 are exposed in the assembled system.

FIG. 7 better shows that the inside surface 706 of the top cover 502 defines an upper trough, and again the upper trough and the lower trough define the interior volume 700. In example cases, to reduce the chance of inadvertently coupling the peristaltic pump 124 (FIG. 1) to the MDU 110, the top cover 502 at least partially occludes the spigot 205 of the MDU 110. In the example shown, the top cover 502 fully occludes the spigot 205 of the MDU 110. Similarly, to reduce the chance of the clinician inadvertently interacting with the valve handle 202 of the MDU 110, the example top cover 502 may also at least partially occlude the valve handle 202 of the MDU 110. In the example shown, the top cover 502 fully occludes the valve handle 202 of the MDU 110. However, because of the example location of the distal end 526 of the top cover 502, the clinician may nevertheless interact with the buttons 148. In order to reduce exposure of the buttons to fluids, a drape may be placed over the entire assembly, starting at the distal end of the length LL and extending over the cover system 500 and the flexible multi-conductor cable 112. The additional drape negates the need to for any cleaning and/or sterility activities after the surgery. The MDU 110 may be immediately moved to the next surgical procedure.

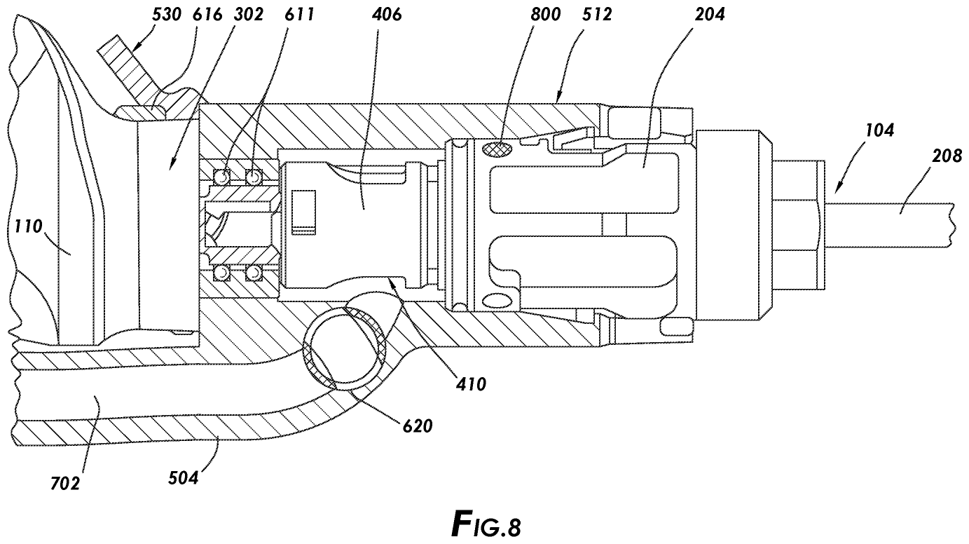

FIG. 8 shows a cross-sectional view of a portion of the cover system in greater detail, and in accordance with at least some embodiments. In particular, FIG. 8 shows a portion of the bottom cover 504, a portion of the MDU 110 (not shown in cross-section) coupled to the bottom cover 504, and a portion of the mechanical resection instrument 104 (not shown in cross-section) coupled to the receptacle 512. FIG. 8 shows a distal end of the example fluid passageway 702 that fluidly couples to interior volume of the receptacle 512. The valve member 620, shown in the closed position in FIG. 8, is disposed in operational relationship to the fluid passageway 702, and the valve handle 530 is shown rotated to the example closed orientation as well.

FIG. 8 also shows additional features. For example FIG. 8 shows the tab 616 extending through and in operational relationship to the slot of the receptacle 302 defined by the MDU 110. Visible in the cross-section are the example O-rings 611 that, in combination with the distal seal area 606 (not numbered in FIG. 6), define the distal seal of the cover system. Also visible in FIG. 8 are the outer hub 204 and the inner hub 406 of the mechanical resection instrument 104. Partially visible in the view of FIG. 8 is the rotating slough chamber 410.

As discussed with respect to FIGS. 3 and 4, the example outer hub 204 of the mechanical resection instrument 104 may comprise one or more magnets, such as magnet 800. The radial location of the magnet 800 with respect to the longitudinal central axis may signify the location of the cutting window 200 (FIG. 2) through the outer tube 208, and may also convey additional information regarding the type and operation of the mechanical resection instrument 104. When the mechanical resection instrument 104 is telescoped directly into the receptacle 302 of the MDU 110, the magnet is placed at the axial position along the longitudinal central axis of a magnetic field sensor (e.g., Hall-effect sensors 322 or 330 (FIG. 3)). However, when using a cover system the magnet is displaced from the magnetic field sensor. Nevertheless, in example cases magnetic field sensor reads or senses a magnetic field associated with the magnet 800. More particularly, in example cases, sensing the magnetic field of the magnet may comprise sensing the magnetic field by way of an elongate structure of ferromagnetic material, the elongate structure having a distal end disposed proximate to the cover receptacle 512 (e.g., aligned axially with the magnet 800), and a proximal end disposed abutting the stationary hub 514 (not shown) of the bottom cover 504 (e.g., aligned axially with the magnetic field sensor in the MDU 110).

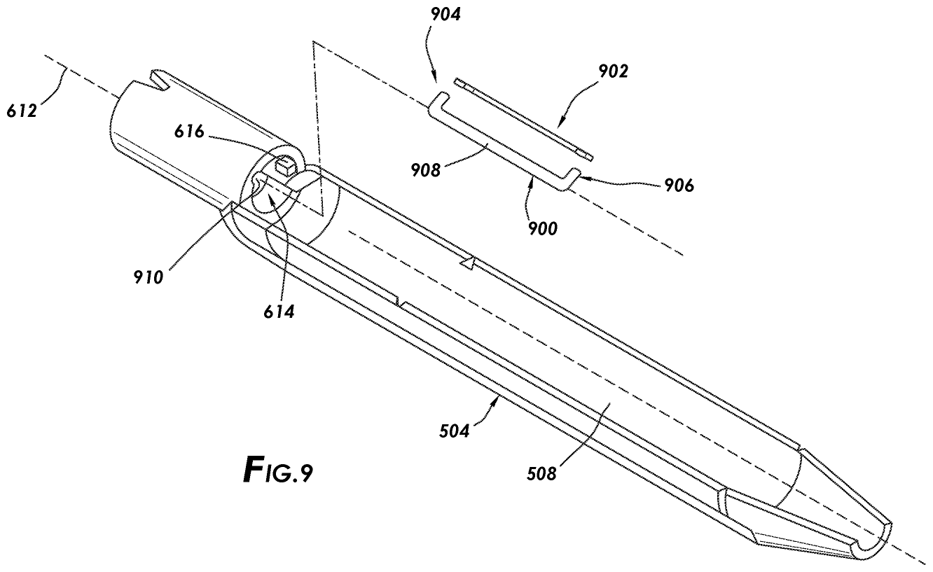

FIG. 9 shows an exploded perspective view of additional components of the cover system, and in accordance with at least some embodiments. In particular, FIG. 9 shows the bottom cover 504, including the lower trough 508 and the through bore 614. FIG. 9 also shows the example system comprises at least one, and in the example case two, strips or elongate bars 900 and 902 of ferromagnetic material. Considering elongate bar 900 as representative, the example elongate bar 900 defines a distal end 904, a proximal end 906, and medial portion 908. The medial portion 908 defines a length measured parallel to the longitudinal central axis 612 (defined by the transmission shaft 600, but the transmission shaft 600 is not shown in FIG. 9). In the example system, the distal end 904 and proximal end 906 define portions shorter than the length of the medial portion 908. In the example shown distal end 904 and proximal end 906 each form an angle (e.g., right angle) with respect to the length of the medial portion 908. In other cases the distal end 904 and proximal end 906 may be omitted. Further in example systems, the elongate bars 900 and 902 are made of ferromagnetic materials, in some cases two more layers of ferromagnetic material separated by an electrically insulating layer, similar to the core material for an electric motor or transformer.

The example bottom cover 504 defines a groove or channel 910 on an inside surface of the through bore 614, where the channel 910 runs parallel to the longitudinal central axis 612. A similar groove or channel for the elongate bar 902 exists on the opposite side of the tab 616, but the channel for the elongate bar 902 is obscured by the tab 616 and related structures, and thus the channel for the elongate bar 902 is not visible in FIG. 9. The example system is assembled by telescoping the elongate bars 900 and 902 through the through bore 614, and then placing each elongate bar in its respective channel. In the example system, when placed in their respective channels, the medial portions (e.g., medial portion 908) are parallel to the longitudinal central axis 612. After the elongate bars are placed in their respective channels, the stationary hub 514 may be telescoped into the through bore 614, and the stationary hub 514 may hold the elongate bars 900 and 902 in place.

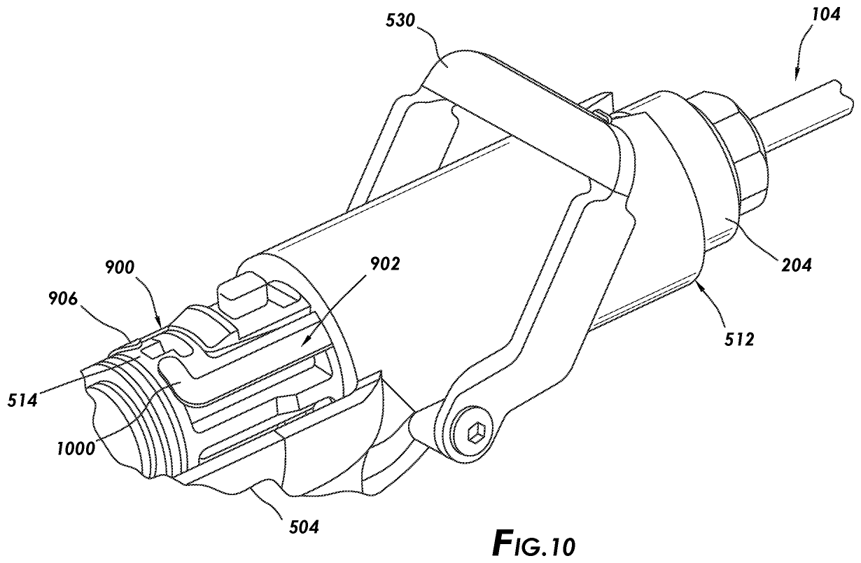

FIG. 10 shows a partial perspective view of the cover system showing placement of the elongate bars in accordance with at least some embodiments. In particular, visible in FIG. 10 is a portion of the bottom cover 504, including the stationary hub 514, the receptacle 512, a portion of the mechanical resection instrument 104, and portions of the valve (e.g., valve handle 530). Further visible in FIG. 10 is the elongate bar 902, and in particular the proximal end 1000 of the elongate bar 902. The distal end of the elongate bar 902 is not visible in FIG. 10, but is discussed in greater detail below. The proximal end 1000 of the elongate bar 902 is disposed on and abuts the stationary hub 514 at a first axial position relative to the longitudinal central axis. The first axial position is proximate to the proximal end of the stationary hub 514, and as discussed more below the first axial position is aligned with a magnetic field sensor of the MDU 110. In some example cases, the MDU 110 may comprise multiple magnetic field sensors on the same side of the receptacle, and similarly the mechanical resection instrument 104 may have multiple magnets associated with its outer hub 204. To accommodate such arrangements, the example system has the other elongate bar 900, with the proximal end 906 only partially visible in FIG. 10. Nevertheless, the proximal end 906 of the elongate bar 900 also disposed on and abuts the stationary hub 514 at the first axial position, though radial positions of the proximal end 906 and the proximal end 1000 relative to the longitudinal central axis are different.

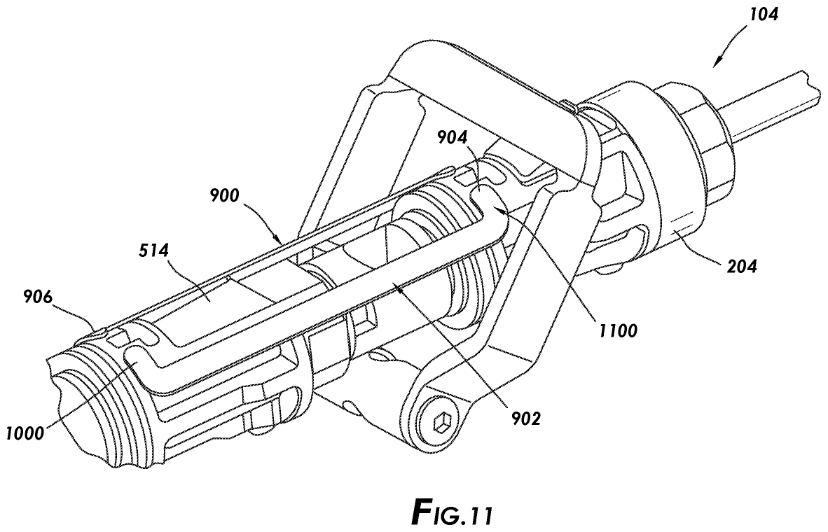

FIG. 11 shows a partial perspective view of a portion of the cover system showing placement of the elongate bars in accordance with at least some embodiments. In particular, FIG. 11 shows the stationary hub 514 coupled to the mechanical resection instrument 104 with the outer cover of the bottom cover 504 removed to better show the relationship between the ends of the example elongate bars 900 and 902 and the remaining components. That is, elongate bar 900 defines its distal end 904 and its proximal end 906. The distal end 904 of the elongate bar 900 resides at a second axial position along the longitudinal central axis of the cover system. When the outer hub 204 of the mechanical resection instrument 104 is telescoped within the receptacle 512 (not shown), the distal end 904 is aligned with a potential location of a magnetic in the outer hub 204. The proximal end 906 of the elongate bar resides at the first axial position, abutting the stationary hub 514. When the MDU 110 (not shown) is coupled to the bottom cover 504 and the receptacle 302 (not shown) of the MDU 110 is telescoped over the stationary hub 514, the proximal end 906 is aligned with a location of a magnetic field sensor in the MDU 110.

Similarly, the elongate bar 902 defines its distal end 1100 and its proximal end 1000. The distal end 1100 of the elongate bar 902 resides at the second axial position along the longitudinal central axis of the cover system, though at a different radial position than distal end 904. When the outer hub 204 of the mechanical resection instrument 104 is telescoped within the receptacle 512 (not shown), the distal end 1100 is aligned with a potential location of a magnetic in the outer hub 204. The proximal end 1000 of the elongate bar 902 resides at the first axial position, abutting the stationary hub 514, though at a different radial position than the proximal end 906. When the MDU 110 is coupled to the bottom cover 504 and the receptacle 302 of the MDU 110 is telescoped over the stationary hub 514, the proximal end 906 is aligned with a location of a magnetic field sensor in the MDU 110. It follows that, in operation, the magnetic field sensor(s) of an MDU 110 are enabled to read magnet(s) associated with the outer hub 204 in spite the additional distance between them created by the use of the cover system.

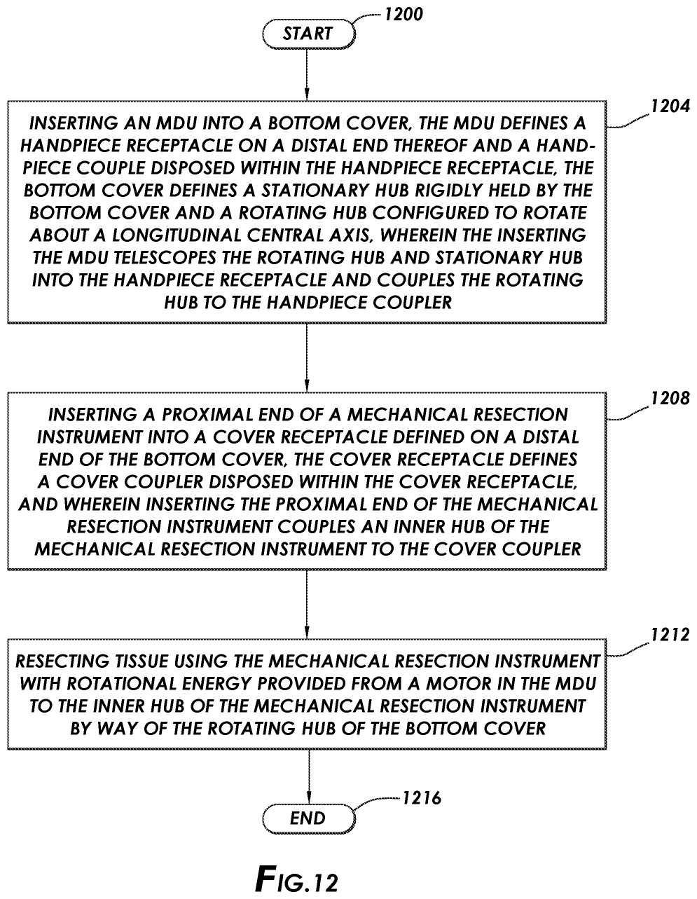

FIG. 12 shows a method in accordance with at least some embodiments. In particular, the method starts (block 1200) and comprises: inserting an MDU into a bottom cover, the MDU defines a handpiece receptacle on a distal end thereof and a handpiece coupler disposed within the handpiece receptacle, the bottom cover defines a stationary hub rigidly held by the bottom cover and a rotating hub configured to rotate about a longitudinal central axis, wherein the inserting the MDU telescopes the rotating hub and stationary hub into the handpiece receptacle and couples the rotating hub to the handpiece coupler (block 1204); inserting a proximal end of a mechanical resection instrument into a cover receptacle defined on a distal end of the bottom cover, the cover receptacle defines a cover coupler disposed within the cover receptacle, and wherein inserting the proximal end of the mechanical resection instrument couples an inner hub of the mechanical resection instrument to the cover coupler (block 1208); and resecting tissue using the mechanical resection instrument with rotational energy provided from a motor in the MDU to the inner hub of the mechanical resection instrument by way of the rotating hub of the bottom cover (block 1212). Thereafter the method ends (block 1216).

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A method comprising:
inserting a motor drive unit (MDU) into a bottom cover, the MDU defines a handpiece receptacle on a distal end thereof and a handpiece coupler disposed within the handpiece receptacle, the bottom cover defines a stationary hub rigidly held by the bottom cover and a rotating hub configured to rotate about a longitudinal central axis, wherein the inserting the MDU telescopes the rotating hub and stationary hub into the handpiece receptacle and couples the rotating hub to the handpiece coupler;
inserting a proximal end of a mechanical resection instrument into a cover receptacle defined on a distal end of the bottom cover, the cover receptacle defines a cover coupler disposed within the cover receptacle, and wherein inserting the proximal end of the mechanical resection instrument couples an inner hub of the mechanical resection instrument to the cover coupler; and
resecting tissue using the mechanical resection instrument with rotational energy provided from a motor in the MDU to the inner hub of the mechanical resection instrument by way of the rotating hub of the bottom cover.

2. The method of claim 1 further comprising placing a top cover over at least a portion of the MDU and coupling the top cover to the bottom cover.

3. The method of claim 2 wherein placing the top cover further comprises at least partially occluding, by the top cover, both a valve handle and a tubing spigot defined by the MDU.

4. The method of claim 2 wherein placing the top cover further comprises fully occluding, by the top cover, both a valve handle and a tubing spigot defined by the MDU.

5. The method of claim 2 wherein the placing the top cover further comprises leaving buttons of the MDU exposed by the top cover.

6. The method of claim 5 wherein leaving the buttons of the MDU exposed further comprises leaving the buttons exposed in a window between a distal end of the top cover and a shoulder defined between the stationary hub and an outer surface of the bottom cover.

7. The method of claim 1 wherein the MDU includes a Hall-effect sensor in operational relationship to the handpiece receptacle, and the method further comprising sensing a magnet field of a magnet disposed in a stationary hub of the mechanical resection instrument.

8. The method of claim 7 wherein sensing the magnetic field of the magnet further comprises sensing the magnetic field of the magnet by way of an elongate bar of ferromagnetic material, the elongate bar having a distal end disposed proximate to the cover receptacle, and a proximal end disposed on a proximal portion of the stationary hub of the bottom cover.

9. The method of claim 1 wherein the MDU further defines a fluid passageway through the MDU, the fluid passageway fluidly coupled to a spigot disposed on the proximal end and an interior volume of the handpiece receptacle, and the method further comprising aspirating resection byproducts through a fluid passageway defined by the bottom cover and fluidly coupled to an interior volume of the cover receptacle.

10. A surgical system comprising:
a bottom cover defining a proximal end and a distal end, the bottom cover comprising:
an exterior surface;
a lower trough defined by an inside surface of the bottom cover;
a cover receptacle disposed at the distal end;
a stationary hub rigidly coupled to the cover receptacle, the stationary hub protruding into the lower trough;
a transmission shaft defining a proximal coupler on a proximal end, a distal coupler on a distal end, and a longitudinal central axis, the transmission shaft extends through the stationary hub such that the distal coupler resides in the cover receptacle, and the transmission shaft is configured to rotate about the longitudinal central axis relative to the stationary hub; and
a motor drive unit (MDU) that defines a motor with a drive shaft, a handpiece receptacle on a distal end of the MDU, and handpiece coupler disposed within the handpiece receptacle and coupled to the drive shaft, the MDU disposed within the lower trough, the handpiece receptacle telescoped over the stationary hub, and the handpiece coupler of the MDU coupled to the proximal coupler of the transmission shaft.

11. The surgical system of claim 10 wherein the bottom cover further comprises:
a spigot on a proximal end of the bottom cover;
a fluid passageway fluidly coupled between the spigot and an interior volume of the cover receptacle; and
a valve comprising a valve member in operational relationship to the fluid passageway and a valve handle rigidly coupled to the valve member.

12. The surgical system of claim 10:
wherein the bottom cover further comprises an elongate bar of ferromagnetic material, the elongate bar defining a first end disposed on the stationary hub at a first longitudinal position relative to the longitudinal central axis, and a second end disposed on an inside surface of the cover receptacle; and
wherein the MDU includes a Hall-effect sensor disposed proximate to the handpiece receptacle at the first longitudinal position.

13. The surgical system of claim 10 further comprising a mechanical resection instrument comprising:
an outer hub coupled to an elongate shaft, the outer hub telescoped within the cover receptacle; and
a rotating hub on a proximal side of the outer hub, the rotating hub coupled the distal coupler within the cover receptacle, the rotating hub is configured to rotate relative to the outer hub about the longitudinal central axis by rotational force imparted by the distal coupler.

14. The surgical system of claim 13:
a Hall-effect sensor disposed proximate to the handpiece receptacle of the MDU, the Hall-effect sensor disposed at a first axial position relative to the longitudinal central axis;
a magnet disposed in an aperture defined in the stationary hub, the magnet disposed within the cover receptacle and at a second axial position relative to the longitudinal central axis; and
an elongate bar of ferromagnetic material, the elongate bar defining a first end disposed on the stationary hub at the first axial position and a second end disposed on an inside surface of the cover receptacle at the second axial position.

15. The surgical system of claim 10 further comprising:
a handpiece spigot on a proximal end of the MDU;
a handpiece fluid passageway fluidly coupled between the handpiece spigot and an interior volume of the handpiece receptacle;
a handpiece valve comprising a handpiece valve member in operational relationship to the handpiece fluid passageway and a handpiece valve handle rigidly coupled to the handpiece valve member;
a top cover coupled to the bottom cover, the top cover comprising:
an exterior surface defining a proximal end aligned with the proximal end of the bottom cover, and a distal end that resides proximally of the stationary hub; and
an upper trough defined by an inside surface of the top cover, the upper trough and the lower trough define an interior volume; and
wherein the top cover at least partially occludes the handpiece spigot and the handpiece valve handle.

16. The surgical system of claim 15 further comprising:
a cover spigot on a proximal end of the bottom cover;
a cover fluid passageway fluidly coupled between the cover spigot and an interior volume of the cover receptacle; and
a cover valve comprising a cover valve member in operational relationship to the cover fluid passageway and a cover valve handle rigidly coupled to the handpiece valve member.

17. The surgical system of claim 10 further comprising:
a pad comprising a button, the pad and button defined by the MDU, and the pad and button proximal from the handpiece receptacle;
wherein the lower trough defines a length measured parallel to the longitudinal central axis from the proximal end to a shoulder defined between the stationary hub and an outer surface of the bottom cover; and
a top cover coupled to the bottom cover over the MDU, the top cover extends from the proximal end of the MDU to a proximal side of the pad and button of the MDU.

18. A device cover for a surgical device, the device cover including a bottom cover defining a proximal end and a distal end, the bottom cover comprising:
an exterior surface;
a lower trough defined by an inside surface;
a cover receptacle disposed at the distal end of the bottom cover;
a stationary hub rigidly coupled to the cover receptacle, the stationary hub protruding into the lower trough;
a transmission shaft defining proximal coupler on a proximal end, a distal coupler on a distal end, and a longitudinal central axis, the transmission shaft extends through the stationary hub such that the distal coupler resides in the cover receptacle, and the transmission shaft is configured to rotate about the longitudinal central axis and the rotation relative to the stationary hub;
a spigot on a proximal end of the bottom cover;
a fluid passageway fluidly coupled between the spigot and an interior volume of the cover receptacle;
a valve comprising a valve member in operational relationship to the fluid passageway and a valve handle rigidly coupled to the valve member; and
an elongate bar of ferromagnetic material, the elongate bar defining a first end disposed on the stationary hub, and a second end disposed on an inside surface of the cover receptacle.

19. The device cover of claim 18 further including a top cover coupled to the bottom cover, the top cover comprising:
an exterior surface defining a proximal end aligned with the proximal end of the bottom cover, and a distal end that resides proximally of the stationary hub; and
an upper trough defined by an inside surface of the top cover, the upper trough and the lower trough define an interior volume.

20. The device cover of claim 18 wherein the lower trough defines a length measured parallel to the longitudinal central axis from the proximal end to a shoulder defined between the stationary hub and an outer surface of the bottom cover, and further including a top cover coupled to the bottom cover, the top cover comprising:
an exterior surface defining a proximal end aligned with the proximal end of the bottom cover, and a distal end, and a length, and wherein the length of the top cover is less than the length of the lower trough; and
an upper trough defined by an inside surface of the top cover, the upper trough and the lower trough define an interior volume.

21. The device cover of claim 18 wherein the valve member and valve handle together circumscribe the longitudinal central axis.
